(19) United States
(12) Patent Application Publication
(10) Pub. No.: US 2025/0320935 A1
(43) Pub. Date: Oct. 16, 2025

(54) A TUBE

(71) Applicant: Coloplast A/S, Humlebaek (DK)

(72) Inventors: Thomas Nesa Gogotic, Hilleroed (DK); Bo Radmer, Hilleroed (DK)

(21) Appl. No.: 18/870,472

(22) PCT Filed: May 31, 2023
(86) PCT No.: PCT/DK2023/050133

§ 371 Date: Nov. 29, 2024

(30) Foreign Application Priority Data
May 31, 2022 (DK) PA202270283

(51) Int. Cl. F16L 11/11; A61M 3/02; F16L 11/04; F16L 11/118

(52) U.S. Cl. CPC F16L 11/111; A61M 3/0254; A61M 3/0279; F16L 11/042; F16L 11/118

(57) ABSTRACT

A kink-free tube is provided. The tube comprises a first part and a second part, both formed by a film material which is a multilayer film material, with an outer layer on opposite sides of at least one inner layer thereby providing enhanced characteristics with respect to burst pressure, anti-kink effect, and opening pressure.

### BRIEF DESCRIPTION OF THE DRAWING

The accompanying drawings are included to provide a further understanding of examples and are incorporated into and a part of this specification. The drawings illustrate examples and together with the description explain principles of examples. Other examples and many of the intended advantages of examples will be readily appreciated as they become better understood by reference to the following detailed description. The elements of the drawings are not necessarily to scale relative to each other. Like reference numerals designate corresponding similar parts.

FIGS. 1A, 1B illustrate what happens, when a tube is subjected to kink.

FIGS. 2A, 2B, 2C illustrate a side view, a cross-sectional view, and another side view of a tube with corrugations.

FIG. 3 illustrates a tube with corrugations.

FIG. 4 illustrates a tube with corrugations and how kink will not happen, even if the tube is bent sharply.

FIGS. 5A to 5D illustrate a tube, where the corrugations initially extend in one direction only.

FIGS. 6A, 6B illustrate a side view and a cross sectional view of a tube where the tube material is longer than the un-stretchable support.

FIG. 7 illustrates a cross-sectional view of a tube with corrugations.

FIGS. 8, 9 illustrate schematically, cross sections of different sheets of a film material.

FIGS. 10-12 illustrate cross-sectional views of tubes.

FIG. 13 illustrates a urinary collection system.

FIG. 14 illustrates an irrigation system.

### DETAILED DESCRIPTION

Examples relate to a tube forming a tube conduit for conducting a liquid from a first end to a second end. The tube has a length in a longitudinal direction, herein defined as extending from the first end to the second end, and a width in a transverse direction, herein defined as transverse to the longitudinal direction. The tube comprises a first part and a second part formed by at least one sheet of a film material. The first part is joined to the second part along at least one longitudinal seal to define the tube conduit between the first part and second part, and at least one of the parts defines corrugations in the form of a sequence of ridges and grooves. The ridges and grooves extend in the transverse direction and the sequence extends in the longitudinal direction. The at least one sheet of a film material is a multilayer film material, with an outer layer on opposite sides of at least one inner layer.

A tube as described above has the advantage that it is very flexible in a transverse direction. The requirement for the film material to both obtain the corrugations and to function as a tube for a liquid flow requires specific material properties.

Since the sheet of a film material is a multilayer film material, the film material can obtain such specific properties both relative to twist, flow, and kink at different pressures of a liquid in the conduit, relative to tensile strength, relative to sealing of two layers during manufacturing, e.g. by welding or adhesive joining of the first part to the second part, and relative to thermoplastic forming of the corrugations. Accordingly, the multilayer film material potentially improves characteristics of the tube with respect to burst pressure, anti-kink effect, and opening pressure, i.e. the pressure of a liquid which is sufficient for pressing the first and second parts away from each other when flowing through the tube conduit.

At least one of the outer layers can be made from a first thermoplastic material and at least one of the at least one inner layers can be made from a second thermoplastic material different from the first thermoplastic material, i.e. one or both outer layers can be made from the first thermoplastic material and one or each of the at least one inner layer can be made from the second thermoplastic material.

Examples of materials which may be useful for the tube are Polyamid (PA), Polyethylene (PE), for example UHMWPE, HDPE, MDPE, LDPE og LLDPE, Polypropylene (PP), Polyethylene terephthalate (PET), Copolymer including Polyethylene (PE) for example including maleic anhydride (PE-MAH), or Ethylene Vinyl Acetate (EVA). The first thermoplastic material can e.g., comprise PE or EVA, and the second thermoplastic material can e.g., comprise HIPS, ABS, PVC, PMMA Acrylic, PC, COC, PP, PE, EVA, or PA. The first thermoplastic material can be selected from the group consisting of Escorene™, Elvax™, and combinations thereof, and the second thermoplastic material can be selected from the group consisting of Escorene™, Grilon CA6™, Surlyn 1650™, and combinations thereof. Additives may be added, for example with the purpose of modifying friction of the tube-material.

These materials may be used either as newly processed materials or as recycled materials. Examples relate to the tube being made at least partly of recycled materials. PET is very common as a recycled material, and this may form at least a part of the material in the tube.

EVA is a material, which is easily stretchable, so this may be easy to process in a vacuum forming process.

Other examples of material are materials including metal, e.g., aluminium, or rubber-materials.

The materials may be used as single-layer or multilayer film or foil-materials and may be combined with each other so as to obtain the desired thickness and flexibility. For example, aluminium may form part as a thin layer in combination with PE or EVA or combinations of PE and EVA and aluminium in a very thin layer. Likewise, recycled PET may form a layer in combination with a more flexible material, for example a layer of EVA or low-density PE.

In an example, the tube is made of thin film or foil material. By thin material is meant a material which is below 90 mu, e.g., below 50 mu or below 20 mu or even below 10 mu. It is contemplated that it may be as thin as 7 mu.

The outer layers and the inner layer can react differently to heating. The inner layer can have a higher melting temperature than the outer layer and it can therefore be easier to form the corrugations in the outer layer by thermoplastic forming at lower temperatures. At the same time, the inner layer can preserve the structural integrity and prevent breakage since it is more resistant to deformation at the temperature where the outer layer can be formed.

The first thermoplastic material can have a melting point of around 60 to 100 degrees Celsius and the second thermoplastic material can have a melting point of around 110 to 140 degrees Celsius.

The at least one inner layer can more specifically be three inner layers or more than three inner layers such as four, five or even more than five inner layers. If the tube comprises three inner layers, the tube comprises five layers including the outer layers, and if the tube comprises five inner layers, the tube comprises a total of seven layer.

The at least three inner layers can comprise an inside inner layer, and middle inner layers on opposite sides of the inside inner layer. The inside inner layer can be made from a material different from the middle inner layers. In this way, the middle inner layers may function as tie-layers binding the inside inner layer to the outer layers. This increases the ability to select materials more freely for the tube and thus better support forming of corrugations in the outer layer and strength in the inside inner layer.

The tube can comprise at least one non-stretchable support extending longitudinally and preventing stretching in the longitudinal direction. This can facilitate a tube which is flexible in a transverse direction, so that it is easily bendable, but at the same time it is un-stretchable in a longitudinal direction. Furthermore, it is possible to stretch the tube in an outer curve of a bend and to butt or compress it in an inner curve of the bend thereby keeping a cross-section across the apex of the bend open for liquid to pass through. This has the effect that the tube will be very unlikely to kink even if it is folded onto itself or curled up in the longitudinal direction.

In the context of this disclosure, a non-stretchable support is defined as a structure which is able to substantially prevent stretching in a longitudinal direction. By substantially prevent is meant that it will not stretch more than 10% during normal use.

In an example, the tube has two non-stretchable supports, particularly longitudinal supports.

In an example, the supports are placed 180 degrees from each other in a circumferential direction around the tube, i.e. one at or integrated in the first part and one at or integrated in the second part.

In an example, the support is made by welding the tube in the longitudinal direction. This means that the tube has one or two longitudinal welds along its length. This is an easy way to create a non-stretchable support in a longitudinal direction. In examples the support comprises three or four welds. The welds may be positioned equidistantly around the circumference but could also be positioned such that a first and second weld are positioned with e.g., 60 degrees between them followed by 120 degrees to the third weld and again 60 degrees to the fourth weld. This leaves again 120 degrees between the fourth and first weld.

The longitudinally extending support can be formed by one of the outer layers, or it can be formed by one or more inner layers, or it can be constituted be a separate element, e.g., attached to an outer surface of one or both outer layers. The longitudinally extending support can be a string or wire melted into or adhesively bonded to the outer surface of the outer layer. If the longitudinally extending support is a separate element, it can be made of a material like the outer layers or identical to the outer layers, like the inner layers or identical to the inner layers, or from a material distinct from the outer and inner layers. The longitudinally extending support can be made from a more rigid or stiff material and particularly from a less elastically deformable material than the outer and inner layers.

Both the first part and the second part can define corrugations in the form of a sequence of ridges and grooves, where the ridges and grooves extend in the transverse direction, and the series extend in the longitudinal direction. The first part and the second part can be identical parts.

The first part and the second part could be separate film layers being joined along at least two longitudinal seals. The at least one and optionally two longitudinal seals can be a thermal-based seal where the outer layer of the first part is thermally bonded to the outer layer of the second part.

The first part and the second part can have different visual expressions allowing a user to determine a difference between two opposite sides of the tube. This may allow the user to determine if the tube is twisted. The different visual expressions could be provided by different colour and/or different texture etc.

The tube may be configured for instillation or drainage of liquid to or from the body of an individual. For this purpose, the tube can comprise an elongated rigid element, e.g., a catheter or a probe forming a rigid conduit from an inlet to an outlet and forming an extension of the tube conduit. In this context, the rigid element has a rigidity exceeding the rigidity of the film material which enables insertion of the catheter through a body opening.

The catheter or probe enables use of the tube e.g., in connection with an irrigation system or for urinary drainage.

An irrigation system typically comprises a reservoir or container for irrigation liquid, an anal probe and tubing connecting those two. The system may also include a pump for pumping the irrigation liquid into the intestines. The tube described herein may be suitable for use in such a system, and it may form an integrated part of such a system, particularly by incorporating the probe at the first end as an integrated, non-detachable, part or a detachable part of the tube, and optionally by incorporating a connector at the second end, again as an integrated, non-detachable, part or a detachable part of the tube. The connector can connect the tube to the pump and reservoir.

A tube as described herein may also be used in connection with urine collection. In systems for urine collection, a user may be provided with either a urinary catheter or a urisheath which leads the urine from the bladder. A urisheath is typically provided with a connector part at its end, to which a connector on a tube can be connected. The tube will then in the other end be connected to a collecting bag.

A catheter is also, typically, provided with a connector part at its end, to which a connector on a tube can be connected. And again, the tube may in the other end be connected to a collecting bag. In case an intermittent urinary catheter is used, the tube may not be connected to a collecting bag, but may rather be provided with an outlet, which is configured for leading the urine directly into a toilet. In this case, the tube may function as an extension tube for an intermittent catheter. For an indwelling catheter, the tube will typically be connected to a collecting bag.

In the context of this description, a tube is an element having a longitudinal extension and which is able to allow liquid to flow through it. A cross-section of the tube may have any shape—and even be closed prior to use.

In this context, kink is defined as an unintended closure of the tube across a diameter, typically due to a bend in the longitudinal direction. When a user handles a rather long tube (e.g., more than 50 cm long), it is easy to inadvertently bend the tube in a longitudinal direction. If such a bend is sharp, then there is a risk that the tube kinks and thereby closes off the inner lumen at the apex of the sharp bend.

The tube as described herein, will not kink and this means that a cross-section across the apex of the bend will always be open as liquid flows through it. So even if the tube is bent sharply, e.g., folded in a longitudinal direction, the tube will not be closed at a cross-section across the apex of the bend.

The apex of the bend will have an outer curve where the material stretches and an inner curve where the material butts.

In an example, the tube has a first width in the transverse direction, which is larger than a second width of the tube in the transverse direction, wherein the first and second widths alternate in a longitudinal direction.

In an example, the tube has a lumen with an interior surface and an exterior surface, wherein the longitudinal support extends radially beyond the exterior surface of the lumen.

Examples relate to a tube with a storage configuration, in which an inner lumen of the tube is substantially closed along the length of the tube, meaning that the tube is substantially air-free in the storage configuration. The tube has a use configuration, in which the inner lumen is open to allow flow of liquid through it.

The transformation from the storage configuration to the use configuration may be done by simply connection the tube to a liquid supply. The pressure from the liquid will automatically open the inner lumen of the tube.

By substantially closed is meant that the inner lumen has a volume in a storage configuration of less than 10% of the volume of the inner lumen in a use configuration.

Such an air-free tube is advantageous in use in an irrigation system because it is undesirable to pump air into the bowels of a user as the irrigation process is initiated. Therefore, in other systems, where the tube includes a volume of air in the storage configuration, the user might have to prime the system, i.e. wait for liquid to have pushed the air out of the tube, prior to initiating the irrigation process.

Examples relate to the tube having a cross-sectional circumference in which a first part around the circumference is convex and a second part around the circumference is concave. The concave part of the circumference extends into the convex part, such that the two parts are close together.

Examples relate to a tube comprising a first and a second half in the longitudinal direction, wherein each first and second half has corrugations in the longitudinal direction and the tube in a storage configuration is collapsed such that the corrugations of the second half is collapsed into the corrugations of the first half.

In an example, the tube is configured for use in an anal irrigation system.

In an example, the tube is configured for functioning as a connection tube connecting a reservoir bag and a urinary catheter.

In an example, the tube is configured for functioning as a connection tube connecting a reservoir bag and a urisheath.

In an example, the tube is configured for functioning as an extension tube for a urinary catheter.

When used for anal irrigation or with urine drainage, it is an advantage if the tube is free of kink. For example, in relation with an anal irrigation procedure, a user might risk that the liquid is prevented from entering the bowels, if the tube kinks. In relation with urine collecting, a leakage might occur, if the tube kinks in such a way that the urine is prevented from being drained into a collecting bag.

Examples relate to a tube where the tube comprises a connector in one end. The tube may also have a connector in both ends, i.e. both in the first and second end.

The connector may be simple friction fit luer-connector configured for connecting to another luer-connector. It may also be a bayonet connector or a threaded connector.

Examples relate to a tube which is configured for being used only once. In other words, the tube is configured for one-time use. In this context, configured for one-time use means that the tube is made of material, which does not withstand being rinsed or otherwise cleaned. This means that the tube may be made of thin recycled material and can be discarded for further recycle following use. This saves material and also relieves the user of a tiresome process of cleaning the tubes and leave them to dry.

### Experiment

Experiments have been Carried Out with

Recipe 1

| | OUTSIDE | MIX in | MIDDLE | MIX in | INSIDE | MIX in |
|---|---|---|---|---|---|---|
| Layer | A 25% | layer | B 50% | layer | C 25% | layer |
| Material | Escorene | 45% | Escorene | 63% | Escorene | 45% |
| | 6224 | 45% | Grilon | 30% | 6224 | 45% |
| | | | CA 6 | | | |
| | Elvax | 10% | Surlyn | 7% | Elvax | 10% |
| | 9619 | | 1650 | | 9619 | |

Recipe 2

| | Layers on the roll | | | | | |
|---|---|---|---|---|---|---|
| | OUTSIDE | MIX in | MIDDLE | MIX in | INSIDE | MIX in |
| Layer | A 25% | layer | B 50% | layer | C 25% | layer |
| Material | Escorene | 60% | Escorene | 78% | Escorene | 75% |
| | 6224 | 30% | Grilon | 15% | 6224 | 15% |
| | | | CA 6 | | | |
| | Elvax | 10% | Surlyn | 7% | Elvax | 10% |
| | 9619 | | 1650 | | 9619 | |

Which provided test results as indicated below:

| | |
|---|---|
| Burst test | Initial opening pressure |

| | |
|---|---|
| Thickness | Bar |

| | | |
|---|---|---|
| 50 | 0.29 | |
| 75 | 0.77 | 0.017 |
| 100 | 0.43 | |
| 50 | | |
| 75 | 0.48 | 0.015 |
| 100 | 0.45 | 0.02 |
| 50 | | |
| 75 | 0.51 | |
| 100 | 0.41 | 0.025 |

This is highly suitable for irrigation and urinary drainage, particularly since the liquid pressure of irrigation liquid and the urinary pressure appear to be sufficient for opening the tube conduit, i.e. for pressing the first part away from the second part. Additionally, the liquid pressure of irrigation liquid and the urinary pressure are lower than the burst test pressure.

### DETAILED DESCRIPTION OF THE DRAWING

Examples, and features of the various exemplary examples described in this application, may be combined with each other (“mixed and matched”), unless specifically noted otherwise.

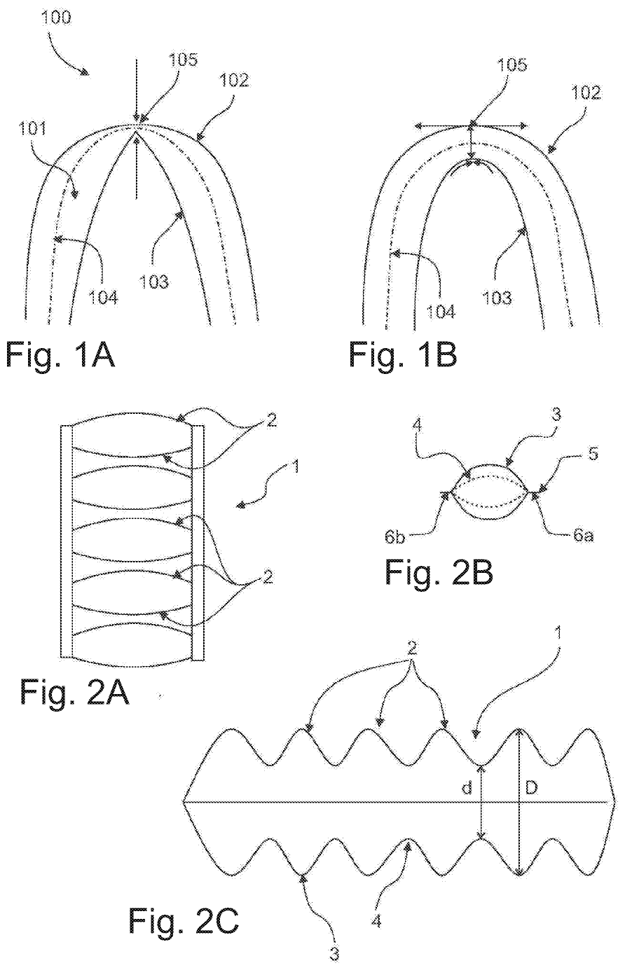

FIGS. 1A, 1B illustrate the problem arising due to kink. In FIG. 1A, a tube 100 is shown. The tube is made of film material 101. When it is bent, the tube will have an outer curve 102 and an inner curve 103 in the bend. A centreline 104 is also indicated in the drawing. The kink occurs, because the inner curve collapses in cusp bend and thereby the cross-section of tube is (almost) closed as indicated at the arrows 105. In FIG. 1B, the situation is different. The outer curve 102 of the tube can stretch as indicated at 105 and the inner curve 103 will compress or butt as indicated at the arrows. This has the effect that the cross-section at the bend remains open.

FIGS. 2A, 2B, 2C illustrate a tube having the ability to stretch in an outer curve and compress or butt in an inner curve. FIG. 2A shows a tube seen from a side, FIG. 2B shows a cross-sectional view of the same tube and FIG. 2C shows the same tube seen from a side perpendicular to FIG. 2A. The tube 1 is made of rather thin material and is corrugated in a longitudinal direction. In FIG. 2C, the corrugations 2 are seen from a side view, so that it is possible to see that a cross-sectional diameter alternates between a smaller diameter d and a larger diameter D in the longitudinal direction. In the illustrated example, the smaller diameter d and the larger diameter D are shown as being consistent through-out the length of the tube. However, examples may be made, where the smaller diameter and/or the larger diameter D varies along the length. An example is shown in FIG. 7.

FIG. 2B illustrates a cross-sectional view of the tube 1. In this figure, it is possible to see that the tube is made of rather thin film-material. Furthermore, the corrugations 2 are illustrated as a top 3 and a trough 4, where the trough 4 is shown in a dotted line indicating that it is not in the same cross-section as a top. The top 3 and the trough 4 can also be seen in FIG. 2C. In this illustration, the larger diameter D is at a top and the smaller diameter d is at a trough.

In FIGS. 2A, 2B, 2C, the non-stretchable support 5 is also illustrated. In this example, the non-stretchable support is in the form of two welds 6a, 6b, one on each side of the tube in the longitudinal direction. This means that the two supports in this example are placed 180 degrees apart in a cross-sectional view of the tube, FIG. 2B.

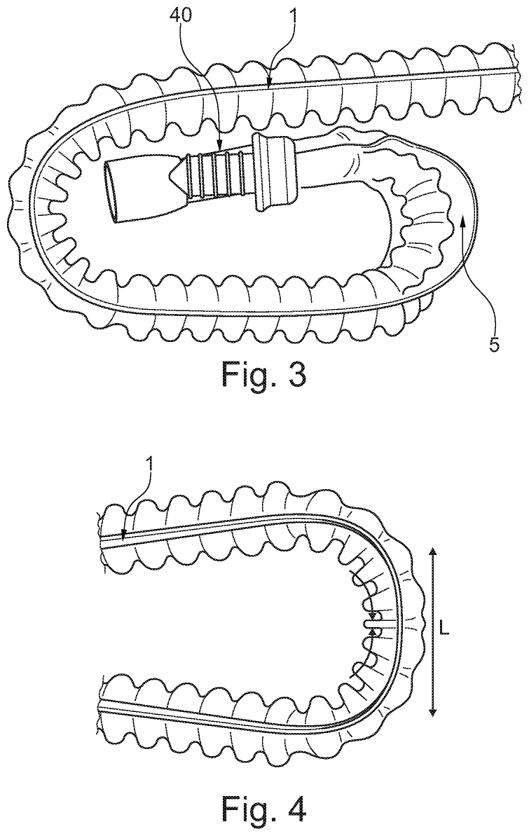

FIGS. 3 and 4 show photos of a tube 1. In FIG. 3, the un-stretchable support 5 is illustrated.

In FIG. 4, it is illustrated that even if the tube is bend sharply, the tube will stretch at an outer curve and butt at an inner curve. This is in this example made possible by the combination of the corrugated tube and the non-stretchable support.

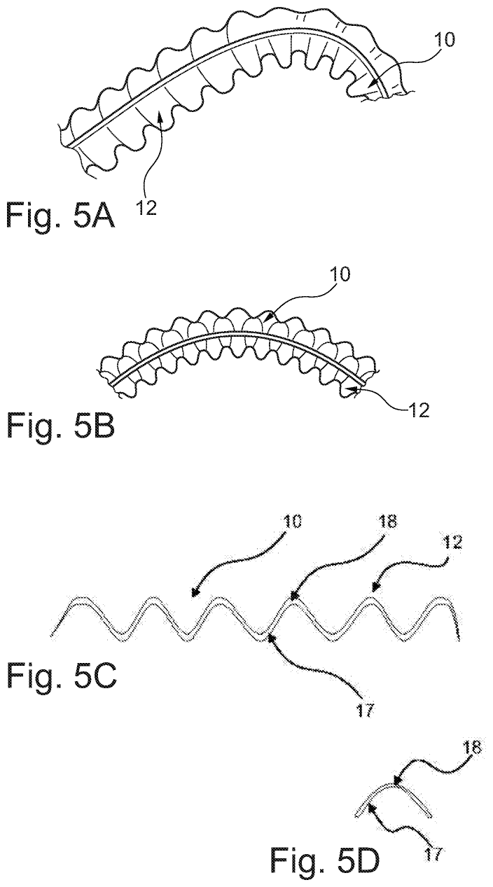

FIGS. 5A, 5B, 5C and 5D illustrate a tube 10 which is manufactured such that the corrugations 12 are made only to one side initially, i.e. such that the corrugations 12 of one half of the tube 17 is collapsed into the corrugations of the other half of the tube 18. This means that the tube has two film layers with very little (or no) air between them prior to use, see FIG. 5A. When the tube is used, i.e. is pressurised by flow of liquid, the tube will un-fold itself as in FIG. 5B, where the corrugations 12 to both sides can be seen. In FIG. 5C, a cross section in a longitudinal direction is seen. The two halves 17, 18 can be seen facing in the same direction such that one of the halves 17 is collapsed into the half 18. In the half 18, the corrugations are convex, and in the half 17, the corrugations are concave.

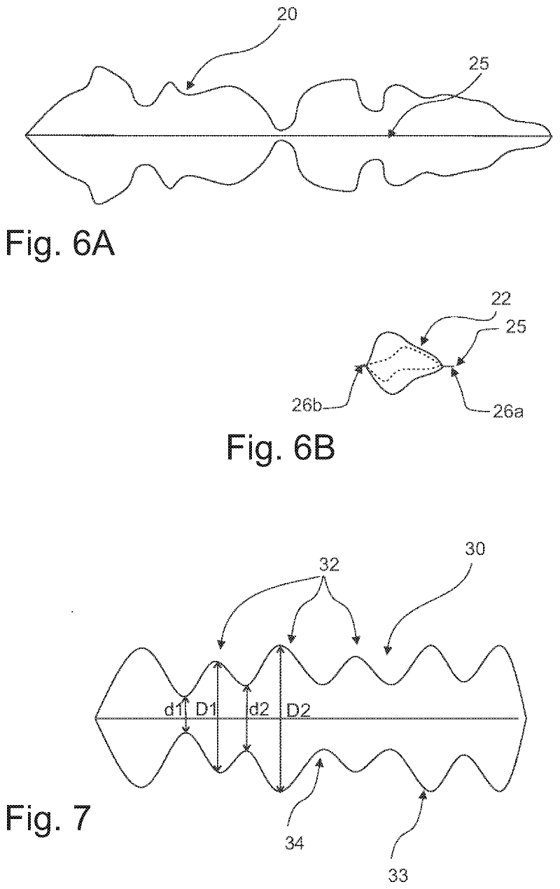

FIGS. 6A, 6B illustrate a tube 20 where the tube material 22 is longer than the un-stretchable support 25. For the corrugated tubes in FIGS. 2 to 5 the tube material is also longer than the un-stretchable support, but in FIGS. 6A, 6B, the longer tube material is in a random way. By random is meant that the tube 20 is not corrugated, but instead the tube material 22 is welded along its length to provide a non-stretchable support 25 in such a way that a length of the tube material in a longitudinal direction is longer than the un-stretchable support 25 in the same longitudinal direction. The non-stretchable support 25 consists of two welds 26a, 26b positioned opposite each other, i.e. with 180 degrees between around the circumference of the tube, see FIG. 6b.

FIG. 7 illustrates a longitudinal cross-section of a tube 30 in which the corrugations 32 varies along its length. As illustrated, the cross section varies between the tops 33 and the troughs 34 from a first small diameter d1 to a first larger diameter D1 back to a second smaller diameter d2 and then to second larger diameter D2 and so forth.

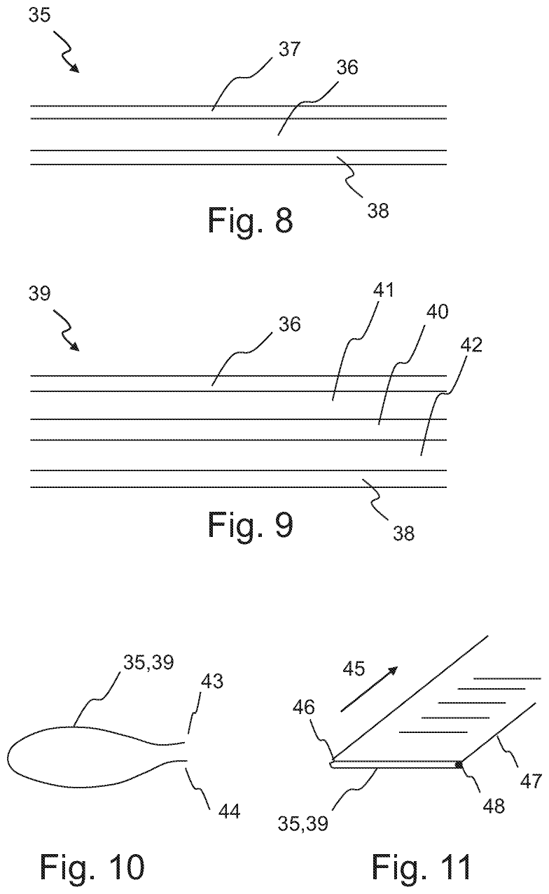

FIG. 8 illustrates schematically, a cross section of a sheet of a film material 35 comprising multilayer film layers, particularly an inner layer 36 and outer layers 37,38 on opposite sides of the inner layer 35. The outer layers are made from a first thermoplastic material and the inner layer 36 is made from a different, second, thermoplastic material. The illustrated multilayer film can be made in a blow moulding process where each layer is blown up against a previous layer. The layers are joined adhesively, e.g., during the blow moulding process where one layer melts onto the previous layer.

FIG. 9 illustrates schematically, a cross section of a sheet of a film material 39 in an example where the tube comprises at least three inner layers, referred to herein as an inside inner layer 40, and middle inner layers 41,42 on opposite sides of the inside inner layer. The inside inner layer 40 is made from a material different from the middle inner layers 41,42.

FIG. 10 illustrates a cross section of the tube in an example where the first part and the second part are formed by a single sheet of a film material 35,39. The single sheet may comprise any number of layers, e.g., 3 or 5 layers as illustrated in FIGS. 8 and 9 or even more layers, such as 7 layers.

To make the first and second parts from a single sheet 35,39 of a film material, the sheet is bent, and the two free edges 43,44 are joined. As illustrated in FIG. 11, the conduit extends longitudinally as indicated by the arrow 45 between the flattened bent edge 46 and the edge 47 constituted by the free edges 43, 44. The two free edges are joined by a welding seam 48 extending along the edge 47.

The first part and the second part could be identical parts, i.e. both having corrugations, or only one of the two parts could have corrugations while the other one could be flat in the sense that is non-corrugated.

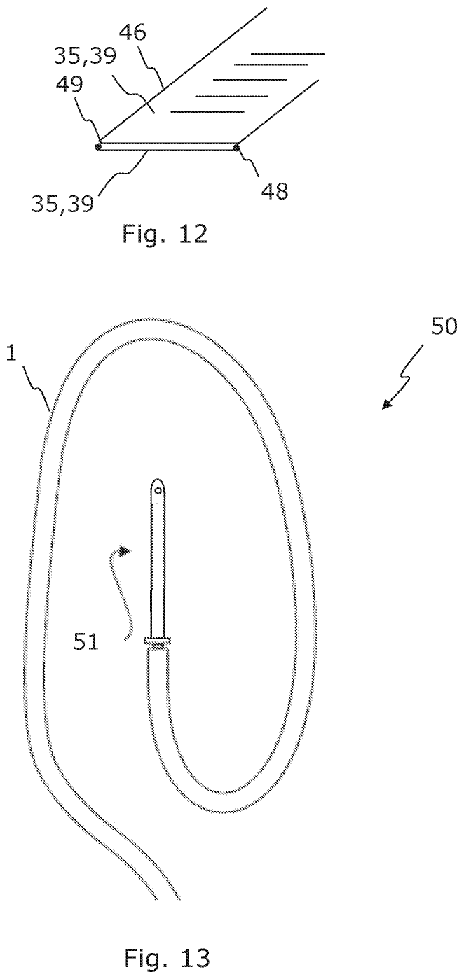

FIG. 12 illustrates a cross section of the tube in an example where the first part and the second part are formed by individual sheets of a film material 35,39. The two individual sheets may comprise any number of layers, e.g., 3 or 5 layers as illustrated in FIGS. 8 and 9 or even more layers, such as 7 layers.

To make the first and second parts from individual sheets 35,39 of a film material, the sheets are arranged in an overlapping configuration and two free edges are joined by two longitudinally extending welding seams 48,49.

FIG. 13 illustrates a urinary collection system 50 comprising a tube 1 as disclosed herein, wherein the rigid element 51 forms a urinary catheter for insertion into a urinary tract, e.g., the urethra during intermittent or indwelling catheterisation to allow drainage of urine through the rigid conduit and the tube conduit. The rigid element 51 could be detachably detached to the tube or it may be fixed irreversibly to the tube.

The catheter is irreversibly or reversibly attached to the first end of the tube and the second end of the tube is optionally irreversibly or reversibly attached to a collection bag. During catheterisation, urine can be drained into the bag, and the anti-kink effect provided by the corrugations prevent blocking of the tube conduit during drainage and thereby prevents a very unpleasant feeling during emptying of the bladder and therefore increases the usability of the urinary collection system. Additionally, the sheet structure of the tube reduces or prevents air in the tube and therefore reduces or eliminates an undesired pumping of air into the bladder during an initial part of the urinary drainage procedure.

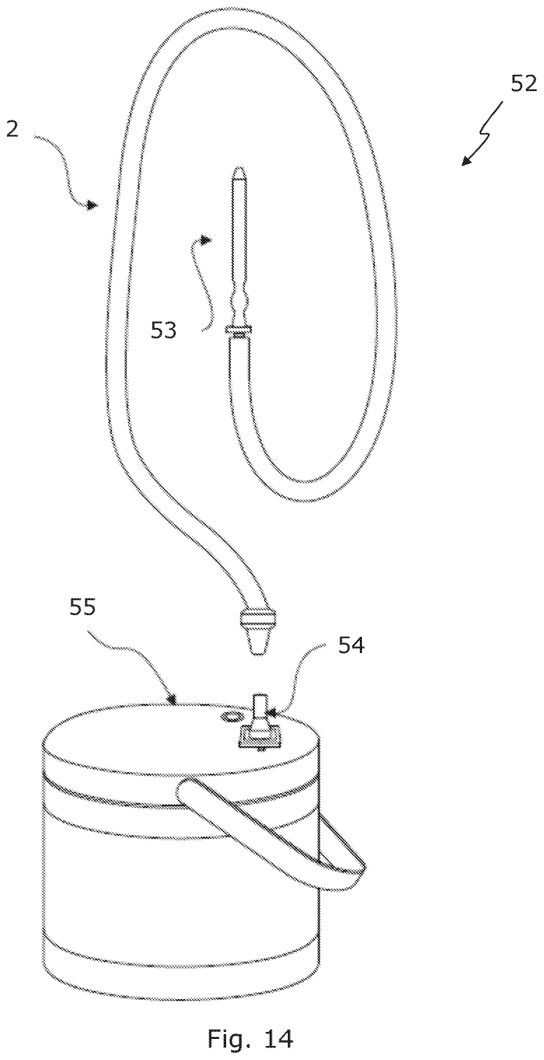

FIG. 14 illustrates an irrigation system 52 for anal irrigation and comprising a tube 1 as disclosed above, a rigid element 53 in the form of a probe, a pump 54 for pumping liquid and being in fluid communication with the tube conduit to thereby enable pumping of liquid through the tube conduit, a reservoir 55 for an irrigation liquid and arranged for the pump to pump the liquid from the reservoir through the tube conduit. In such a system, the anti-kink effect provided by the corrugations prevent blocking of the tube conduit during irrigation and therefore increases the usability of the irrigation system. Additionally, the sheet structure of the tube reduces or prevents air in the tube and therefore reduces or eliminates an undesired pumping of air into the body during an initial part of the irrigation procedure.

The probe is irreversibly or reversibly attached to the first end of the tube and the second end of the tube is irreversibly or reversibly attached to the pump or to the reservoir. For this purpose, the first and second ends of the tube may comprise a connecting structure, e.g., a threaded connection structure.

1. A tube forming a tube conduit for conducting a liquid from a first end to a second end, wherein the tube has a length in a longitudinal direction defined as extending from the first end to the second end, and a width in a transverse direction defined as transverse to the longitudinal direction, wherein the tube comprises a first part and a second part formed by at least one sheet of a film material, the first part being joined to the second part along at least one longitudinal seal to define the tube conduit between the first part and second part, and at least one of the parts defining a sequence of ridges and grooves, the ridges and grooves extending in the transverse direction, and the sequence extending in the longitudinal direction, wherein the at least one sheet of a film material is a multilayer film material, with an outer layer on opposite sides of at least one inner layer.

2. The tube according to claim 1, wherein at least one of the outer layers is made from a first thermoplastic material and at least one of the at least one inner layer is made from a second thermoplastic material different from the first thermoplastic material.

3. The tube according to claim 2, wherein both outer layers are made from the first thermoplastic material.

4. The tube according to claim 2 or 3, wherein each of the at least one inner layer is made from the second thermoplastic material.

5. The tube according to any of claims 2-4, wherein the first thermoplastic material comprises PE or EVA, and the second thermoplastic material comprises HIPS, ABS, PVC, PMMA Acrylic, PC, COC, PP, PE, EVA, or PA.

6. The tube according to any of claims 2-5, wherein the outer layers and the inner layer reacts differently to heating.

7. The tube according to any of claims 2-6, wherein the first thermoplastic material has a melting point of around 60 to 100 degrees Celsius and the second thermoplastic material has a melting point of around 110 to 140 degrees Celsius.

8. The tube according to any of the preceding claims, wherein the at least one inner layer comprises at least three inner layers.

9. The tube according to claim 8, wherein the at least three inner layers comprises an inside inner layer, and middle inner layers on opposite sides of the inside inner layer.

10. The tube according to claim 9, wherein the inside inner layer is made from a material different from the middle inner layers.

11. The tube according to any of the preceding claims, wherein the tube has at least one longitudinally extending support configured for preventing stretching in the longitudinal direction.

12. The tube according to claim 11, wherein the longitudinally extending support is formed by one of the outer layers.

13. The tube according to any of the preceding claims, wherein both the first part and the second part define a sequence of ridges and grooves, the ridges and grooves extending in the transverse direction, and the series extending in the longitudinal direction.

14. The tube according to claim 13, wherein the first part and the second part are identical parts.

15. The tube according to any of the preceding claims, wherein the first part and the second part are separate film layers being joined along at least two longitudinal seals.

16. The tube according to any of the preceding claims, wherein the at least one longitudinal seal is a thermal-based seal where the outer layer of the first part is thermally bonded to the outer layer of the second part.

17. The tube according to any of the preceding claims, wherein the first part and the second part have different visual expressions.

18. The tube according to any of the preceding claims comprising an elongated rigid element formed integrally with or connectably to the first end, the rigid element forming a rigid conduit from an inlet to an outlet and forming an extension of the tube conduit, the rigid element having a rigidity exceeding the rigidity of the film material.

19. The tube according to any of the preceding claims comprising a pump for pumping liquid and being in fluid communication with the tube conduit to thereby enable pumping of liquid through the tube conduit.

20. The tube according to claim 19, comprising a reservoir for containing liquid and wherein the pump is integrated in the reservoir.

21. The tube according to claim 20, wherein the at least one sheet of a film material is connected to the reservoir.

22. An irrigation system comprising a liquid reservoir and a tube according to claim 19, the pump being in fluid communication with the reservoir to enable irrigation with liquid pumped by the pump through the tube conduit.

23. The irrigation system according to claim 22, comprising a probe forming a probe conduit from an inlet to an outlet and forming an extension of the tube conduit, the probe having a rigidity exceeding the rigidity of the film material.

24. A urinary collection system comprising a tube according to claim 18, wherein the rigid element forms a urinary catheter for insertion into a urinary tract to allow drainage of urine through the rigid conduit and the tube conduit.

25. The urinary collection system according to claim 24, comprising a collection bag formed integrally with or connectably to the second end.

26. A method of making a tube having a first end and a second end, wherein the tube has a length in a longitudinal direction defined as extending from the first end to the second end, and a width in a transverse direction defined as transverse to the longitudinal direction, wherein the tube comprises a first part and a second part formed by at least one sheet of a film material, the first part being joined to the second part along at least one longitudinal seal and at least one of the layers defining a sequence of ridges and grooves, the ridges and grooves extending in the transverse direction, and the series extending in the longitudinal direction, wherein the at least one sheet of a film material is a multilayer film material, with an outer layer on opposite sides of at least one inner layer, the method comprising thermally influencing the outer layer of at least one of the first part and second part while forming the ridges and grooves by blow moulding.

27. The method according to claim 26, wherein the ridges and grooves are formed in the at least one sheet of a film material prior to the joining of the first part to the second part.

28. A method of using a tube having a first end and a second end, wherein the tube has a length in a longitudinal direction defined as extending from the first end to the second end, and a width in a transverse direction defined as transverse to the longitudinal direction, wherein the tube comprises a first part and a second part formed by at least one sheet of a film material, the first part being joined to the second part along at least one longitudinal seal and at least one of the layers defining a sequence of ridges and grooves, the ridges and grooves extending in the transverse direction, and the series extending in the longitudinal direction, wherein the at least one sheet of a film material is a multilayer film material, with an outer layer on opposite sides of at least one inner layer, the method comprising allowing the first part and a second part to be in close contact, and to move apart by pressure of liquid being flushed longitudinally through the tube.
